(19) United States
(12) Patent Application Publication
(10) Pub. No.: US 2014/0188009 A1
(43) Pub. Date: Jul. 3, 2014

(54) CUSTOMIZABLE ACTIVITY TRAINING AND REHABILITATION SYSTEM

(71) Applicants: Belinda Lange, Aliso Viejo, CA (US); Albert ("Skip") Rizzo, Los Angeles, CA (US); Sheryl Flynn, Altadena, CA (US); Chien-Yen Chang, Alhambra, CA (US); Sebastian Koenig, Aliso Viejo, CA (US); Eric McConnell, San Francisco, CA (US)

(72) Inventors: Belinda Lange, Aliso Viejo, CA (US); Albert ("Skip") Rizzo, Los Angeles, CA (US); Sheryl Flynn, Altadena, CA (US); Chien-Yen Chang, Alhambra, CA (US); Sebastian Koenig, Aliso Viejo, CA (US); Eric McConnell, San Francisco, CA (US)

(73) Assignee: UNIVERSITY OF SOUTHERN CALIFORNIA, Los Angeles, CA (US)

(21) Appl. No.: 13/937,038

(22) Filed: Jul. 8, 2013

Related U.S. Application Data
(60) Provisional application No. 61/668,829, filed on Jul. 6, 2012.

(51) Int. Cl. A61B 5/11

(52) U.S. Cl. CPC A61B 5/1128
USPC 600/595

(57) ABSTRACT

A system may include a calibration module that measures a range of limb and trunk movements made by a human subject during a calibration phase of the system. A customization module may customize pre-determined performance criteria based on the measured range of limb and trunk movements. An activity task presentation module may present an activity task to the human subject for performance during an activity phase. An activity assessment module may assess the human subject's performance of that activity task during the activity phase based on the pre-determined performance criteria.

### CROSS-REFERENCE TO RELATED APPLICATION

This application is based upon and claims priority to U.S. provisional patent application 61/668,829, entitled “Flexible System To Tailor, Deliver, And Analyze/Track Exercise Training And Rehabilitation,” filed Jul. 6, 2012, attorney docket number 028080-0765. The entire content of this application is incorporated herein by reference.

### STATEMENT REGARDING FEDERALLY SPONSORED RESEARCH

This invention was made with government support under Grant No. W911 NF-04-D-0005, awarded by the Army Research Office, and Grant No. H133E080024, awarded by the United States Department of Education. The government has certain rights in the invention.

### BACKGROUND

1. Technical Field

This disclosure relates to activity training and rehabilitation systems.

2. Description of Related Art

The use of video games for exercise and rehabilitation has expanded rapidly over the past five years. Virtual reality (VR) and game-based technology may improve motor skill rehabilitation for a range of functional deficits. Video games and VR applications may also be used as effective exercise tools for strength training, sport training, and weight loss. Video games may present a task that demands focus and attention, motivates the user to move, and provides the user with a sense of achievement, even if the user cannot perform that task in the real world.

However, the exercise and rehabilitation system may be expensive and usable only in research clinics. A clinic or home-based system may also not be easy to set-up, use, and/or maintain. It may also be limited in its ability to accurately track the user and provide appropriate feedback to meet real world training goals. The game-play may also be too fast, require the player to perform inappropriate and potentially unsafe movements, and provide scoring that does not further therapeutic functional outcome goals and/or that is demeaning of the player (e.g., feedback that the user failed the task or is are ‘unbalanced’). These games may also not have the accuracy needed for tracking and guidance, nor appropriate feedback for exercise training.

### SUMMARY

A system may include a calibration module that measures a range of limb and trunk movements made by a human subject during a calibration phase of the system, a customization module that customizes pre-determined performance criteria based on the measured range of limb and trunk movements, a movement task presentation module that presents a movement task to the human subject for performance during an activity phase, and an activity assessment module that assesses the human subject's performance of that movement task during the activity phase based on the pre-determined performance criteria.

The activity assessment module may have a viewing window within which it monitors movement of the human subject and that locks onto the human subject. This may enable the activity assessment module to not erroneously confuse movement of another person or persons within the viewing window as movement of the human subject.

The customization module may adjust the pre-determined performance criteria during the activity phase so as to encourage the human subject to continue the movement task and to discourage the human subject to perform inappropriate and potentially unsafe movements.

The system may include a data storage module that stores information indicative of the human subject's movement during the calibration phase and/or the human subject's performance of the movement task during the activity phase. A playback module may play back the stored information after the calibration phase and/or activity phase.

The activity assessment module may determine the velocity of limb and/or trunk movement made by the human subject during the activity phase and utilize this determined velocity in the assessment.

The activity assessment module may determine a range of movement made by the human subject during the activity phase and utilize this determined range of movement in the assessment.

The calibration module may determine a maximum range of movement made by the human subject during the calibration phrase. The activity assessment module may assess the human subject's performance of the movement task during the activity phase to have been satisfactory when the determined range of movement made by the human subject during the activity phase meets or exceeds a pre-determined portion of the maximum determined range of movement that is less than 100% of the maximum determined range of movement. The activity assessment module may also assess the human subject's performance and provide incentive for movement tasks that exceed 100% of the maximum determined range of movement.

The movement task presentation module may track the movement of the human subject during the activity phase, display an avatar that moves in substantial synchronism with the human subject during the activity phase, and smoothen the movements of the displayed avatar so as to substantially stop momentary lapses in the tracking of the movement of the human subject from causing sudden jumps in the movement of the displayed avatar.

The movement task presentation module may adjust the height and limb length of the avatar to reflect the height and limb length of the human subject.

The movement task presentation module may receive information concerning the movement of other human subjects that are not at the same location as the human subject and, for each such other human subjects, display an additional avatar that moves in substantial synchronism with the other human subject.

The movement task may be an exercise task.

A non-transitory, tangible, computer-readable storage media may contain a program of instructions that cause a computer system running the program of instructions to perform one, any combination, or all of the functions of any of the versions of the system that have been described herein.

These, as well as other components, steps, features, objects, benefits, and advantages, will now become clear from a review of the following detailed description of illustrative embodiments, the accompanying drawings, and the claims.

### BRIEF DESCRIPTION OF DRAWINGS

The drawings are of illustrative embodiments. They do not illustrate all embodiments. Other embodiments may be used in addition or instead. Details that may be apparent or unnecessary may be omitted to save space or for more effective illustration. Some embodiments may be practiced with additional components or steps and/or without all of the components or steps that are illustrated. When the same numeral appears in different drawings, it refers to the same or like components or steps.

FIG. 1 illustrates an example of a virtual reality (VR) system.

FIG. 2 illustrates an example of how a VR system may be used for clinical training and/or assessment.

FIG. 3 illustrates an example of a VR system and how components of the system interact with each other.

FIG. 4 illustrates an example of a relationship between input devices and a user's representation (avatar) within a VR system.

FIG. 5 illustrates an example of a calibration method.

FIGS. 6A-D illustrate an example of a replay interface that allows a review of whole body motor behavior in real time.

FIGS. 7A-C illustrate graphs of example joint trajectories.

FIG. 8 illustrates an example of a game set in a jewel mine.

FIG. 9 illustrates an example of a user calibration step in which a user strikes a default pose.

FIG. 10 illustrates an example of a calibration step in which a player's range of motion is mapped.

FIGS. 11A and 11B illustrate examples of avatar representations in a game. FIG. 11A illustrates an example of an upper torso skeleton. FIG. 11B illustrates an example of only a player's hands, in this case represented as two squares.

FIG. 12 illustrates an example of data that may be recorded during a game that may include a player profile and time to complete in-game tasks.

FIG. 13 illustrates an example of a calibration screen in a soccer game.

FIG. 14 illustrates an example of playing a soccer game.

FIG. 15 illustrates an example of a one player version of a card-match game.

FIG. 16 illustrates an example of a two player version of a card-match game.

FIGS. 17A-17C illustrate examples a shoulder strengthening exercise in a shoulder game.

FIG. 18A-18G illustrate an examples of a series of performance feedback.

FIG. 19A-19D illustrate an example of changes to the environment.

FIGS. 20A-B illustrate examples of how a game may be tailored at several points.

FIGS. 21A-B illustrate two levels in an example Star tour game.

FIG. 22A-22D illustrate examples of a shopping game. FIGS. 22A-22B illustrate examples of shopping tasks, and FIGS. 22C-22D illustrate examples of task scenes.

FIGS. 23A-B illustrate an example of a boundary box formed by a user that serves as a calibrated area of movement.

FIG. 24 illustrates an example of a game task in which a user is asked to control a circle or shaped object on a screen.

FIG. 25 illustrates example of a game task in which a user is asked to control a balloon on a screen.

FIGS. 26A-B illustrate an example of a game task in which a user is asked to control an ice cream cone on a screen.

FIGS. 27A-B illustrate an example of multiple body-tracked avatars across a network being rendered in a single display.

FIG. 28 illustrates an example of modules in an exercise system.

FIG. 29 illustrates an example of a non-transitory, tangible, computer-readable storage media containing a program of instructions.

### DETAILED DESCRIPTION OF ILLUSTRATIVE EMBODIMENTS

Illustrative embodiments are now described. Other embodiments may be used in addition or instead. Details that may be apparent or unnecessary may be omitted to save space or for a more effective presentation. Some embodiments may be practiced with additional components or steps and/or without all of the components or steps that are described.

Games used for exercise and rehabilitation may focus on specific movement goals and provide appropriate feedback to the user. The feedback may provide the player with useful information about their actions and improve and motivate skill acquisition, without reducing player morale. To accomplish this, the appropriate movements may be tracked, recorded, and analyzed. The level of challenge of the task may be changed by the player, trainer, or therapist to allow the game to be challenging enough to motivate users to improve, but easy enough for the task to be performed. These criteria may be integrated into games in the exercise, rehabilitation and game design fields.

Several affordable input devices, VR applications, and display solutions may be integrated to provide interactive training and assessment for people undergoing neurological and orthopedic rehabilitation and for people undergoing regular exercise programs. The VR applications may be based on clinical evidence and experience of clinicians who are closely integrated in the development process. An iterative design and development approach may be taken in which feedback from clinicians and key end users (patients, exercise groups, trainers, clinicians etc.) is gathered and integrated into the applications. There may be a well-organized structure between the VR applications, input devices, and output devices.

Table 1 lists input and output devices that have been utilized in laboratory experiments over the past six years. Many of these system features may be interchangeable.

TABLE 1
List of VR systems that have been used in Laboratory

| | | Interface between | | | Interface between | | |
|---|---|---|---|---|---|---|---|
| | | the program and | VR Apps/Data | Game | the program and | | |
| Prototypes | Output Devices | the display | Analysis tool | Engine | the input devices | Input Devices | |
| Balloon | Head Mounted | DirectX | Specifically | Ogre3D | User defined - | 1. | Motion Trackers |
| Ice Cream | Display (HMD) | OpenGL | designed | Panda3D | Specifically | | Flock of birds |
| Chinese Character | Panochamber | Specifically | by team | Gamebryo | designed | | eMagin |
| Dance Game | (360 degree display) | designed | | Unity3D | by team | | Inertial cube |
| Jewel Mine | Mono Monitor | (C++ code or | | Unreal | | | Camera Tracking |
| Shoulder Game | Stereo Display | C# code) | | OpenGL | | | Color LEDs |
| Soccer Game | Projector Display | | | XNA | | | Camera Tracking |
| Star tour | | | | | | | Infrared reflector |
| Penguin | | | | | | | Primesense/Kinect Camera |
| Jewel Mine | | | | | | | Wiimote + motion plus |
| Card Match | | | | | | | Kinect |
| | | | | | | 2. | Force Feedback devices |
| | | | | | | | Novint Falcon |
| | | | | | | | Omni |
| | | | | | | 3. | Cyper gloves |
| | | | | | | 4. | Wiifit Board |
| | | | | | | 5. | Breathing devices |
| | | | | | | 6. | DDR Pad |

### Flexible VR System Design and Implementation

Object-oriented analysis and design principles may be used to develop exercise systems.

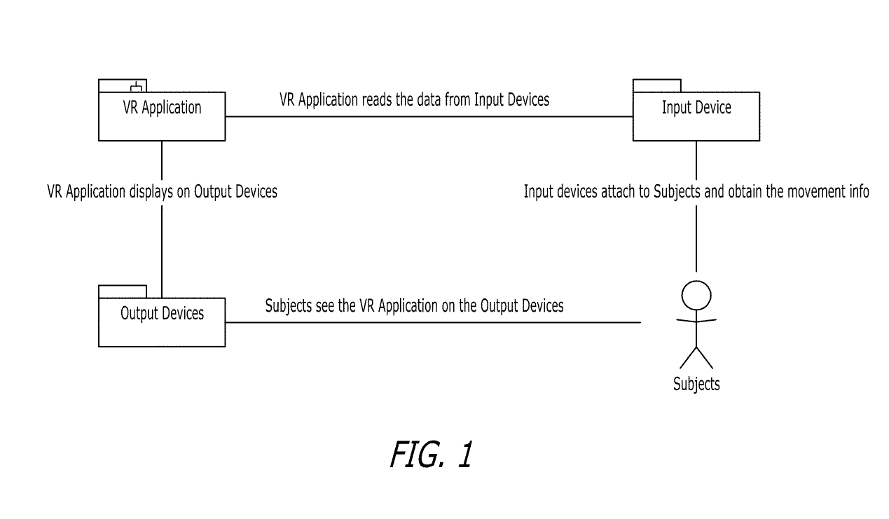

FIG. 1 illustrates an example of a virtual reality (VR) reality system. The user may choose input devices to access the VR application, such as one or more cameras, position sensors, keyboards, and/or mice. An output device such as a display may display feedback of any user interaction.

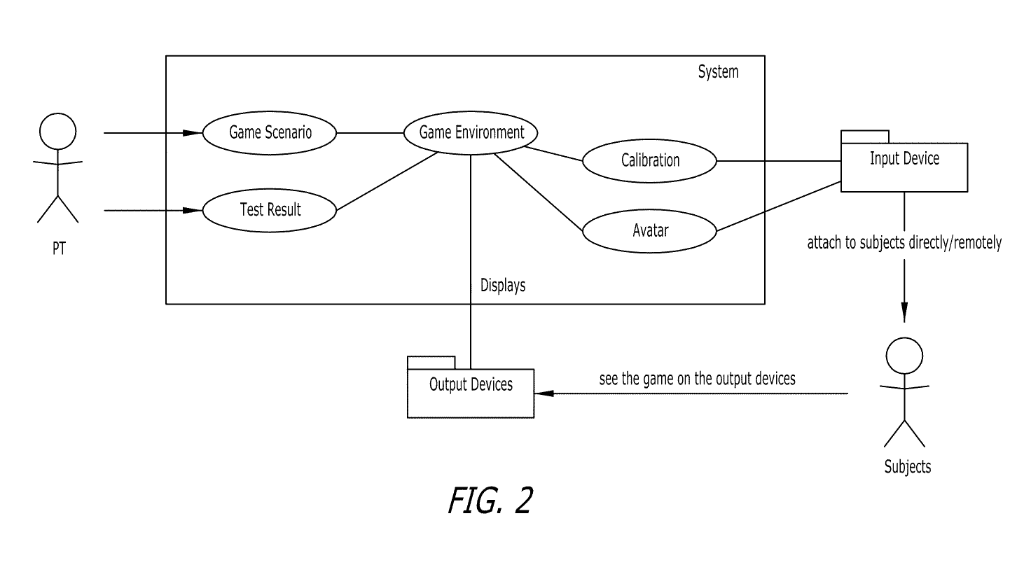

FIG. 2 illustrates an example of a how a VR system may be used for clinical training or assessment. A user and/or trainer/therapist may interact with the system by choosing a game scenario and interpreting test results. The patient may interact with the application via input and output devices. In a VR application, the components may include a game environment, game scenario, calibration tool, avatar, and test results.

The game environment may be the visual representation of the game scenario as displayed by the output device.

The game scenario may represents a task context as defined by a user, trainer, clinician or third party (depending on the specific purpose of the clinical application) and may provide the basis on which the game is played.

The calibration tool may be used to set up the game scenario based on exercise or rehabilitation goals that are suitable for a user's individual level of ability. The calibration functionality may allow the player to work at a level that is appropriate for them; not a generalized level that could be too easy or too difficult for them.

The avatar may imitate the user and his/her movements within the game environment.

The test results may include raw data of signals sent from an input device and the user performance within the game environment as it relates to the game scenario. The user, trainer, clinician, or third party may use the test results to evaluate the user's progress within and across sessions and to gain more quantitative information about the user's condition. The information from the test results may be used within the application to adjust the difficulty level automatically (based on a predefined set of criteria defined by the user and/or clinician/third party).

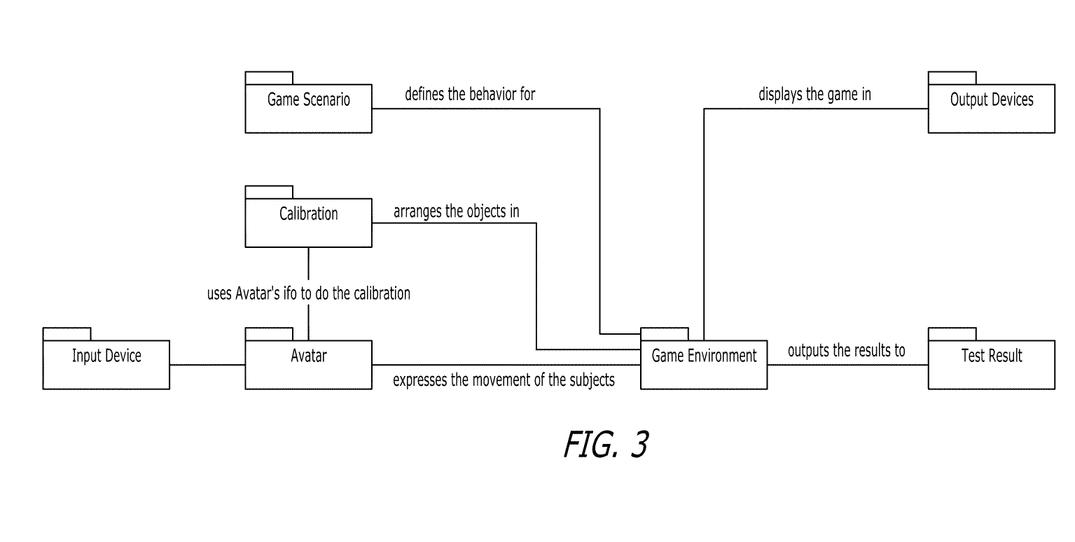

FIG. 3 illustrates an example of a VR system and how components of the system interact with each other. FIG. 3 is a class diagram of the structure of a VR health-care system and shows the relationship between components depicted in FIG. 1.

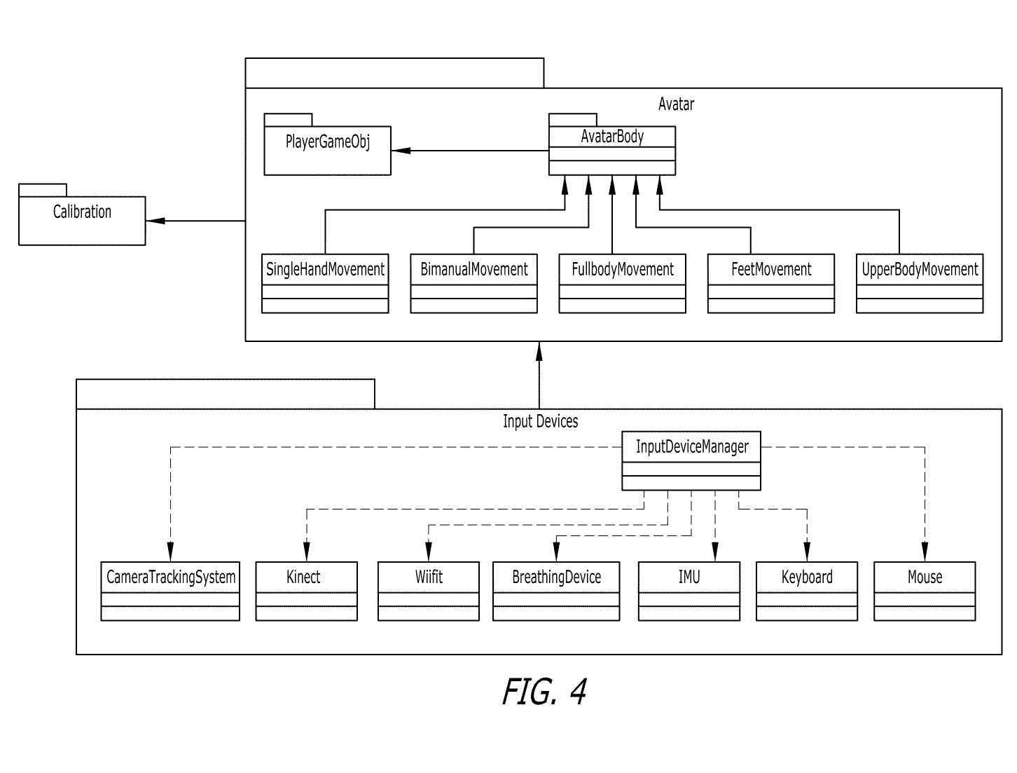

FIG. 4 illustrates an example of a relationship between input devices and a user's representation (avatar) within a VR system. An InputDeviceManager may allow all applications to be controlled by listed input devices and may relay input data to the system via an AvatarBody component. Depicted are example supported input devices and how they may obtain user information to provide an interface with the VR system. The avatar component may access the input device to relay any incoming hardware signal to the VR system.

### Features of Game-Based Exercise Rehabilitation/VR Application for Exercise and/or Rehabilitation

The game-based interactive exercise and/or rehabilitation tools may be based on one or more of the following design principles:
1) Calibration of user interaction to a user's level of ability;
2) Customization of content based on a user's goals and/or ability; and/or
3) Data collection and presentation that are appropriate for expert instructors, users and their goals.

Examples of these concepts and their implementation are now described. Also described are examples of specific applications that use the features to demonstrate how these applications may be tailored for individualized level of ability and interaction goals using different hardware devices.

### Calibration of User Interaction to User's Level of Ability

The application may allow a user to perform an initial calibration in which key task elements are set-up in positions that are appropriately challenging for the user.

The user may be asked to perform actions to determine their movement limitations, such as reaching to determine range of motion and/or weight shifting to determine a user's balance. This may be performed by tracking the user's limb and trunk movements and comparing the movement of the limbs in relation to trunk movement.

A user's movement limitations may be quantified as metrics which are provided by the input device. For example, locations in 3D space of the user's range of limb and trunk motion may be quantified using a Microsoft Kinect. Weight distribution as the user stands may be tested using a Wii Fit Balance Board.

These metrics—referred to as calibrations—may be saved by the application in a database, either locally or remotely.

A calibration may be set by the user remaining at a limit of their movement range for a pre-determined time, such as for 2 seconds. This may trigger the current data from the input device in this extreme position to be saved as a calibration point.

Several calibrations may be saved for each user to accommodate different user goals under different circumstances, such as while seated, standing, and/or with additional rehabilitation tasks.

Each task may then use a calibration specific to the user and rehabilitation goal to set task elements to the appropriate difficulty level, such as placing a reaching target at a previously calibrated position in 3D space.

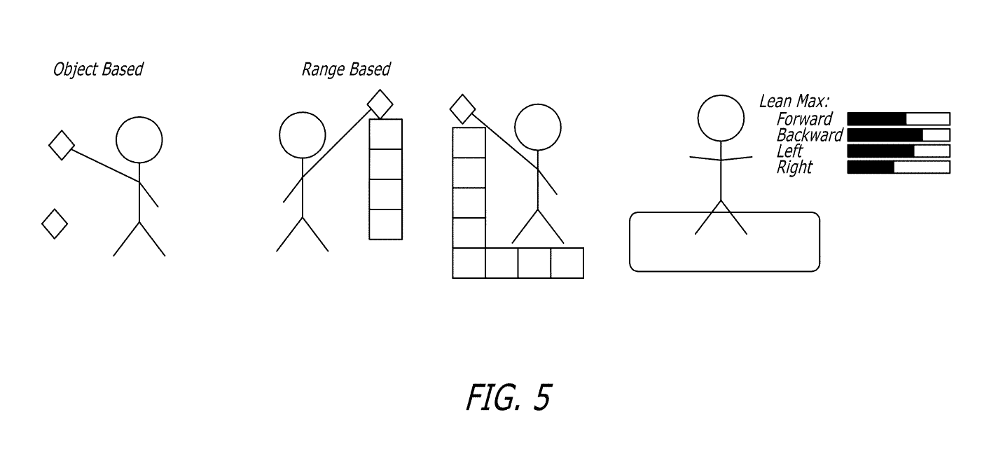

FIG. 5 illustrates an example of a calibration method. Calibrations may be derived based on the nature of the input device.

For example, a calibration may be object based. A calibration may be set as a discrete position in 3D space which represents the appropriate reach of the user towards a target stimulus. This position may then be used to represent task-specific target items.

A calibration may be range based. A calibration may be determined as a set of maximum trunk and limb ranges, each along a relevant direction, such as a maximum forward reach, left reach, and/or right reach, to form an area or volume within which task-specific targets are then displayed. The targets may be placed at a randomly selected point in the range area or a randomly selected section in a scalable grid that exists in the calibrated range.

### Customization of Content Based on User's Goals

An expert instructor (clinician/third party) or the user may select different tasks that are appropriate for achieving the user's specific or individualized goals.

Each task may target specific skills and may be aimed at an overall level of ability for the particular user.

The overall goals/tasks may be chosen by the user and/or a coach, clinician, third party and/or guide to provide a tailored exercise program for the user.

An expert instructor and/or the user may further customize each task by enabling or disabling features that are appropriate to the user's goals and level of ability, such as turning off skeleton visualization and/or pushing limits of targets slightly beyond initial calibration.

Multiple tasks may be combined during a single session to form a comprehensive training regime.

Tasks may be selectively made available to the user by an expert in order to foster user progression towards a goal.

The goals/tasks may use the user's calibration profile to ensure the game-play/task is provided at an appropriate level of challenge for the user.

Each task may contain previously set calibrations to make them available to users with varying levels of ability.

Tracking may be ‘locked’ to one user, or transferred between users.

During gameplay, an object may be moved dynamically. This can provide additional difficulty or bring the object closer to the user's reach.

A target object may be skipped if the user temporarily cannot reach the location of the target. This may be done by moving ahead to the next target in a task list. A user may not get a “score” by skipping an object, nor may the target object get destroyed. However, the game may go on to target the next object. Doing so on the last target object in that task's target set may advance the player to the next target set.

The scene graphics may be altered in real time to provide a different environment for the task, such as different backgrounds and/or different game objects. The calibration points may be used to determine an object's position during the game. The user may dynamically change the entire environment by swapping art assets. Pressing the arrow keys may completely change the current environment by disabling all environmental art, enabling new environmental art, destroying current virtual target objects, and instantiating new objects at the same calibration point. Changing the art assets may not change the task progress. For example, if a user was had successfully completed six of a total of eight target, they may still only have two objects left to complete after the art swap.

Improved 3D Sensing Tracking within Applications May be Effectuated by Using Devices Such as Microsoft Kinect and/or Primesense Hardware.

Smoothing: The position of all joints tracked by the Microsoft Kinect may be collected and imported into rehabilitation applications through an external dll. Before applying data to objects within the rehabilitation application, a smoothing procedure may be applied to handle cases of tracking loss. For example, the Microsoft Kinect provides a confidence value for each tracked joint position. Whenever this confidence value indicates that the position of a joint has not been tracked confidently in the current processing frame (i.e. confidence=0), the position of the tracked joint may not be updated. Instead, the last tracked position with a high level of confidence may be used until a new location is obtained at a high confidence level. This procedure may avoid inappropriate behavior of virtual limbs and the unwanted activation of game mechanics associated with such movement.

Skeleton Transfer: When using software that requires a “calibration pose” to begin to track a user, such as OpenNI, the application may allow a second user to calibrate for the primary user. This approach may be used, for example, if a user cannot perform a “calibration pose.” The application may allow the second user to perform the “calibration pose” to begin tracking. The system may save the skeleton information of the user in the computer memory after the user performs the calibration pose. When tracking is transferred between users, a button may be pressed on a keyboard, such as the “T” key. The system may load the previously saved body position/skeleton tracking information from the computer memory to assign this information to the user. The “assign” function may be made available from software such as OpenNI.

Tracking upper limb only: For people in a seated position or in a wheelchair, lower limb tracking may be inaccurate or difficult. To implement tracking of the upper limb only, the system may read the information of the trajectories of the upper limb joints in every frame, such as Head, Torso Center, Left Shoulder, Right Shoulder, Left Elbow, Right Elbow, Left Hand, and Right Hand. This may be read, for example, from an OpenNI, Microsoft Kinect SDK, and/or Unity3D plugin. The system may update these results to the avatar in the game after calculating the orientation between the two bones. One may be from Shoulder to Elbow. The other may be from Elbow to Hand.

Skeleton locking: In order to maintain tracking of one user when another person or other people are in view of the Kinect, skeleton locking may be implemented. This may be important because, when other people are positioned within the tracking volume of the Kinect, the Kinect can switch from one user to another without notice. Locking of the skeleton may be implemented within the plugin for the application. Each tracked user may be assigned an ID and only the first registered user may be recognized and updated by the system. All other tracked users may not be updated within the system. If a second or third person is in the scene, the tracking may be transferred using a key press on a keyboard.

### Data Collection and Presentation that are Appropriate for Expert Instructors, Users and their Goals

Data about a user's full-body motion in 3D space—referred to as motion data—may be collected throughout each task. Motion data may be combined with temporal information and events and outcomes that are specific to each task and the enabled features. All motion data, outcomes, and events may relate back to the calibration and may be presented to the expert instructor and user visually through an avatar.

Metrics to quantify user performance may be calculated based on motion data and saved events and displayed to the expert instructor and user.

During each frame that the application is executed, skeletal information of the tracked user may be appended to a text file. This information may include the 3D world space, quaternion, confidence, scale of all joints, and/or time stamp for each frame. It may also record the event information from the game play, such as when the game starts, when relevant objects are displayed, and when the player completes a task. After finishing the game, the task name, time and date, score, avatar style, and response time for each trial and/or for the whole task session may be saved to a local hard drive. Also, if internet connectivity is available, all results may be saved to a database for further evaluation and analysis.

The saved information may then be uploaded to a database where it may be parsed and stored in a relational or non-relational database, such as MySQL. The game engine Unity may be used to develop a web program to allow users, including system administrators, clinicians, and patients, to review the game play results via a PC, Mac, or any mobile device.

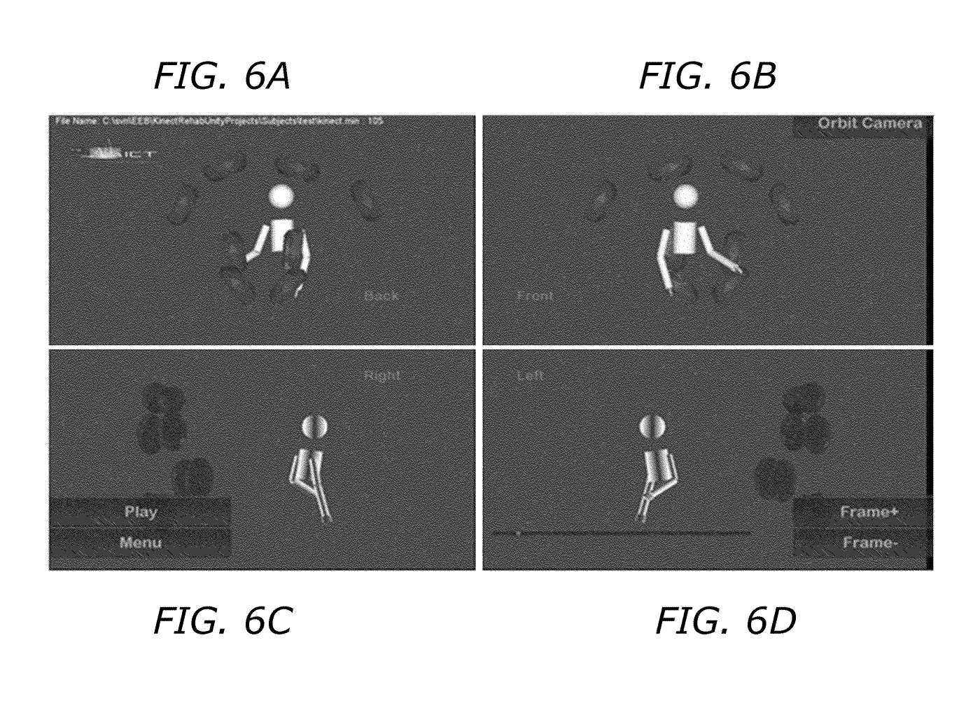

FIGS. 6A-D illustrate an example of a replay interface that allows a review of whole body motor behavior in real time.

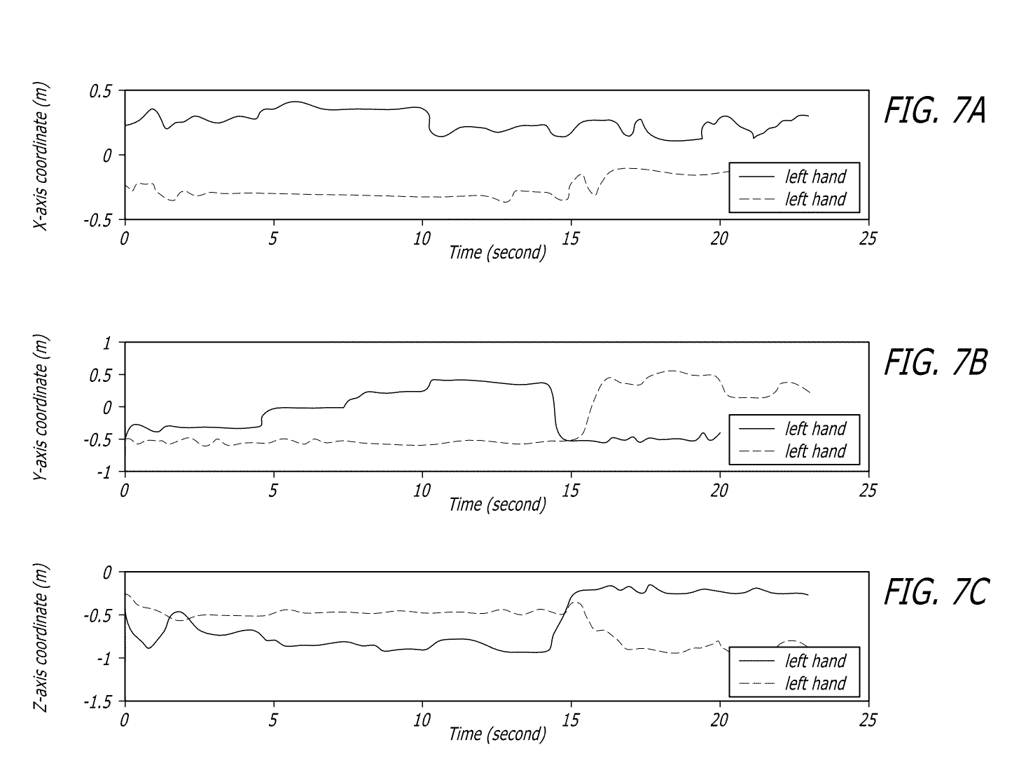

FIGS. 7A-C illustrate graphs of example joint trajectories. The replay may also contain the graphics of all joint trajectories, as shown in FIGS. 7A-C. These two visualization features may assist clinicians to evaluate the performance of patients' motor behavior. The program may calculate maximum speed and acceleration of patient hand movement as the quantitative features to represent the performance of the movement. The program may also compute the orientation between upper-arm and forearm and track the distances between human subject's torso and limbs or their hips and elbows to validate the task. For example, when the patient reaches the object, clinicians may want to know if the human subject extends their hand to hit the object or used another way to reach the target. By searching the database, the history of the calibration results and quantitative features may be obtained, through which clinicians, trainers and/or the human subject may evaluate the human subject's performance.

The game-play data may be recorded throughout the task/game/session and saved to a file on the computer. The data collected during game play can be used to alter the tasks/games in real time or in subsequent sessions. The data collected during game play may be saved within a database and analyzed to provide detailed feedback on performance.

The following is a summary of possible ways to implement features in a game-based application:

1. Identify the Exercise or Task
a. Identify the body parts to be tracked
   i. i. e.g., an Exercise might only need to track the shoulder
   ii. e.g., an Exercise or Task might require tracking of weight shift
b. Identify the constraints
   i. Identify and outline the range of motion each body part will be moved in
c. Identify the measurements
   i. tracking the 3D position
   ii. tracking the rotation
   iii. tracking distance from or to another object
   iv. tracking weight placement
d. Identify the feedback
   i. i.e.g. the elbow is too far from the body
   ii. how would a clinician and/or trainer assess and respond to the player's motion

2. Identify the Device
a. What needs to be measured
   i. i.e.g. balance based exercise would use the WiiFit
   ii. is there need to track the player's skeleton?
b. Get driver to connect Device to computer
c. Write .dll to interface the Device hardware to the game engine

3. Create the player's Calibration
a. Convert the Exercise constraints and measurements into computer data
   i. i.e.g. create a ranges of 3d points that describe correct movement
   ii. define the points the player might have to reach
   iii. define the balance the player must maintain
b. Create a Calibration
   i. identify the player's threshold for each one of those constraints
   ii. save that information in a file
   iii. save correct points or a range of points that constitute correct motion

4. Create the Game
a. Create one or a series of environments suitable to the target audience
   i. a younger, tech savvy crowd wants immersive environments
   ii. an older, more conservative crowd wants less distractions
   iii. the art in the environment can, and should, be interchangeable
b. Duplicate the exercise in the game world
   i. imitate the real world exercise in the game world
c. Use the Calibration to adjust the Game to the player's individual specifications
   i. manipulating object placement
   ii. defining Exercise constraints
d. Give feedback Exercise accordingly
   i. using what was identified as a key measurement
   ii. this should to be tracked real time
   iii. can be game related, like a score, or literal such as text
e. Create an overlay to provide abstraction
   i. turn the exercise into a game
   ii. do not modify the Exercise
   iii. this is a combination or gameplay, sound, environment, game flow
   iv. this can be a simplistic or complex

5. Save Data and Replay
a. Save game session data to a file upon completion
   i. feedback given
   ii. measurements
   iii. calibration used
b. Save a replay of the player playing the Game
   i. save frame by frame information of the player's actions during the game
   ii. load those actions into the game for a replay
c. Data saving may be an important part of an exercise game

### Example

### Jewel Mine Game Design and Implementation

A JewelMine game may be developed using the game engine Unity (http://unity3d.com). Unit may be used to integrate game scenarios, for calibration, and to display test results, analysis, and avatar movement. As a game engine, Unity may provide many features (e.g. underlying math functions, graphics rendering) which may be used in JewelMine.

Game assets (Jewels, environments, avatar) may be created using Google SketchUp Pro and Autodesk Maya. The game may integrate the Microsoft Kinect input device via an OpenNI-Unity Wrapper (open-source https://github.com/OpenNI/UnityWrapper) and UnityKinectPlugin.dll, (http://groups.google.com/group/unitykinect/msg/a1631444739a95c8), an open source plugin to communicate with the Microsoft SDK API to access the Microsoft Kinect Camera. The communication with these plugins may be managed by a class named “HumanSensor,” which is an equivalent of “InputDeviceManager” as seen in FIG. 4.

Further, AviFile.dll (open source plugin http://www.codeproject.com/Articles/7388/A-Simple-C-Wrapper-for-the-AviFile-Library) may be used to capture AVI videos of the user interacting with the game. Alternatively, a color image screenshot may be recorded for each calibration position through, for example, the Microsoft Kinect sensor's RGB video stream, During the game play, the Microsoft AVI API may be used to collect and convert the RGB video images of the sensor to an *.avi file.

A basic interaction with a database may be explored using a php script to communicate with the server.

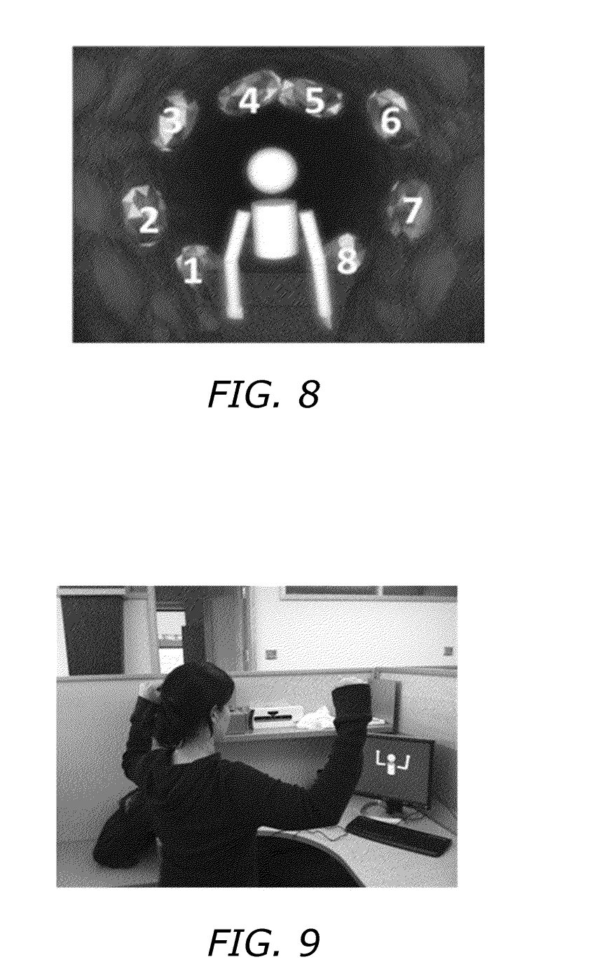

FIG. 8 illustrates an example of a game set in a jewel mine. The player's character may be situated in the center of the mine shaft with eight gems placed around them in a ring.

Game Overview: The game may involve a jewel mine where the player assumes the role of a miner who rides a railroad cart down a mine shaft and gathers jewels (target objects) from the shaft walls. The shaft may be uniformly cylindrical with eight jewels (target objects) arranged in a ring with the player's avatar centered in the middle of the screen (FIG. 8). In order for the player to successfully gather all the jewels (target objects), they may need to reach out from the center of the screen and touch each jewel (target object) individually with their hand.

Calibration: The game may begin with a calibration step, which may map the range of motion of the player's upper limbs.

FIG. 9 illustrates an example of a user calibration step in which a user strikes a default pose. The player may strike a default pose and the player's upper torso, arms, hands and head may be represented on the screen.

After striking a default calibration pose for a few seconds (FIG. 9), the player's skeleton may be captured and tracked without any attachment of devices or markers.

The length of the cylinders for the chest and arms may be scaled to match each player's specific anatomy.

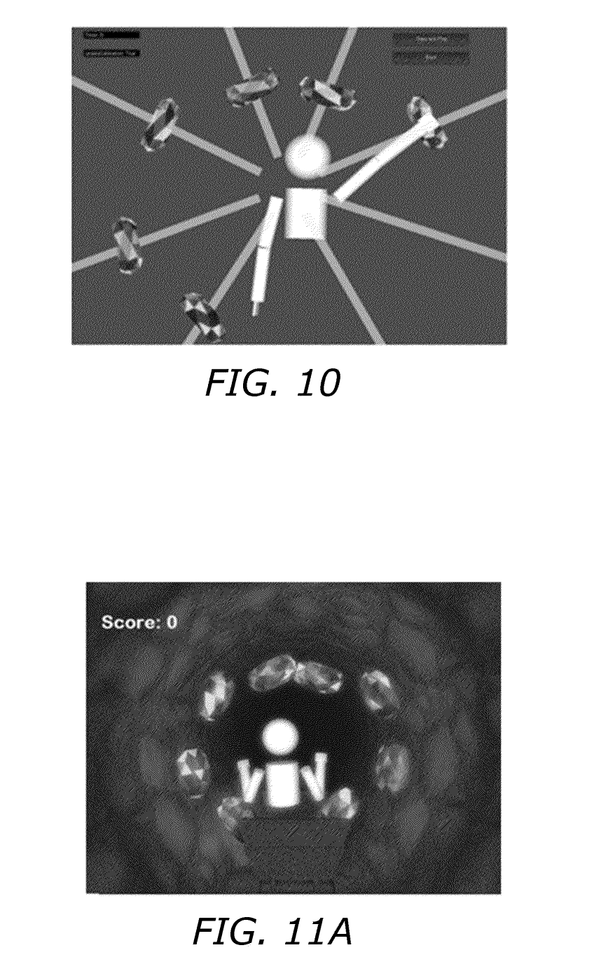

FIG. 10 illustrates an example of a calibration step in which a player's range of motion is mapped. To map the individual player's range of motion, the player may move their arms as far as possible in 3D space forward and outward in an arc over eight radiating lines. This step may be guided by a clinician to encourage movements that are appropriate for the player and specific therapy goals. As the player holds their hand at the intersecting point of each line, a jewel may be placed at the point of intersection (FIG. 10). Upon completing the placement of each jewel, the positions may be saved and loaded into the game itself.

This calibration screen may allow the user to save and store a specific individualized profile to meet the needs of players with different levels of ability and different rehabilitation goals. A number of profiles may be saved for one person, depending on how the clinician/third party wants them to interact with different games. For example, the clinician may want a client to work on reaching across their midline in standing with both left and right hands. They could save a profile with the client using just their right hand to calibrate and save another profile using just the left hand. They could also have the same client reach out of their base of support to work on standing balance, and therefore save another profile using both left and right hands and encourage full reach to limit of stability at each of the calibration points.

Previously saved calibration files may be loaded from a menu screen. This may save the client from having to calibrate the system each time.

Gameplay: In the game, all eight jewels (target objects) may be present and visible in a series of rings that extend as the player moves through the mine shaft. Individual jewels (target objects) may glow to indicate when they may be gathered. The order in which the jewels (target objects) glow may be controlled by three different pre-defined patterns that the clinician/third party may select before a session begins: Sequential pattern, Simon pattern, and Sam pattern. During the Sequential pattern, jewels (target objects) may light up one by one. The player's goal may be to collect the gems as they light up. During the Simon pattern, the jewels may light up in the classic “Simon” game pattern, and the player may need to remember the sequence in which the jewels (target objects) light up and touch them in that order. The Sam pattern may be a modified “Simon” pattern where the recall task is removed and the jewels (target objects) remain lit until the player collects them. The Sequential and Sam patterns may each have multiple difficulty settings. There may also be an option for clinicians to load their own pattern. A range of other tasks may also be included.

The user may select which game/task they want to play. The user may also select easy, medium, or hard for each of these patterns. This may affect how objects in the game will be targeted and interacted with. This may also add cognitive layers or keep the game relatively simple.

Example of some tasks include:
Sequential: The target object may be lit in order according to their task file
Sam: The target objects may be lit in the order they need to be targeted and stay lit
Simon: The target objects may be lit in the order they need to be targeted and then the user must touch objects in the order they lit up (memory task)

A task file may contain which target objects need to be targeted each round. This may be one object or all eight. The game may light up objects for Sam and Simon by either activating a glow halo or changing the mesh material of the object itself.

The user next may select which calibration they want to use. A profile may have multiple calibrations. These calibrations may be object based or range based as previously stated. Users may also re-calibrate a calibration if a specific calibration becomes too easy or too difficult.

The calibration (x,y and z game position in relation to the Kinect) may dictate how the target objects will be manipulated within the game. The target objects may translate according to a calibration, creating an in-game representation of what the user's abilities are. Every target object may be in the same position it was calibrated in, assuming that the user is standing in the same position, in relation to the tracking device (for example the Kinect sensor), as when it was calibrated.

The tasks may be altered in real time to change the level of challenge during the session/game play. This may be done based on the degree to which a user successfully performs each task.

A target object may be moved in one of six directions, (up, down, left, right, forward, backward). Moving the object may be achieved through key presses on the keyboard number pad (up—8, down—2, left—4, right—6, forward—0, backward—5). Multiple key presses may register at once, giving diagonal movements. Generally, the target object may be the only one that can be moved. The user may now have to collide with the object's new position.

### Avatar Representation

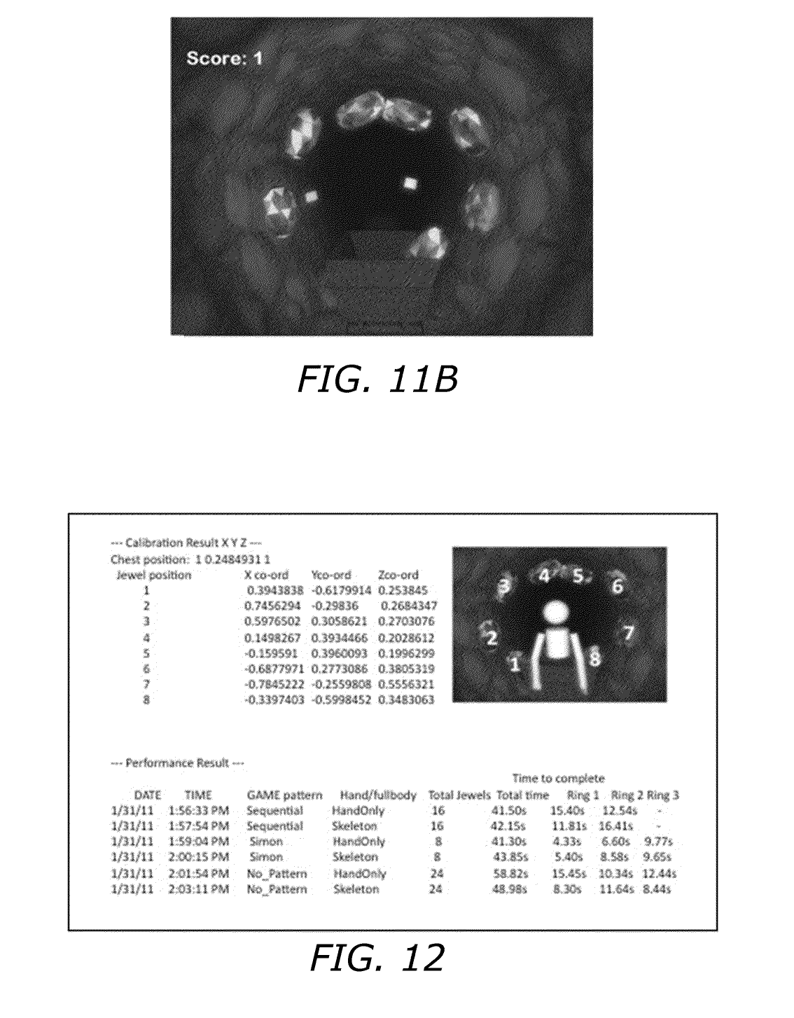

FIGS. 11A and 11B illustrate examples of avatar representations in a game. FIG. 11A illustrates an example of an upper torso skeleton. FIG. 11B illustrates an example of only a player's hands, in this case represented as two squares.

Before starting the game, the clinician may be given the option of displaying the player's avatar as either a full torso with arms and head or simply displaying the hands (FIG. 11A and FIG. 11B). This avatar representation may allow the clinician to control how much feedback the player receives about their body position during the game. The full upper torso may be tracked within both representation settings. However, the hands only representation may only provide visual representation of the hand points of the skeleton.

FIG. 12 illustrates an example of data that may be recorded during a game that may include a player profile and time to complete in-game tasks.

Data Recording: The player's range of motion calibration settings may be saved as a player profile and may be loaded in subsequent sessions. Additionally, the duration of time it takes patients to complete each jewel ring task and the total time it takes to complete the game may be recorded and reported (FIG. 12).

### Example

### Soccer Game Design and Implementation

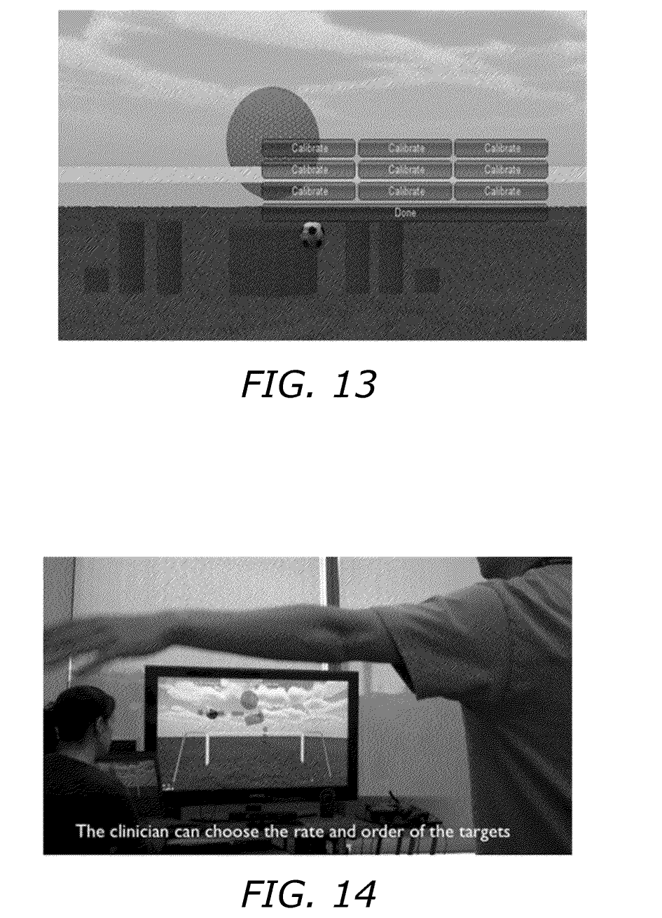

FIG. 13 illustrates an example of a calibration screen in a soccer game. FIG. 14 illustrates an example of playing a soccer game. During the calibration, the user may reach to nine calibration points on the screen. These points may be saved. The calibration points may be used within the game (FIG. 14. right) to determine the path of the soccer balls toward the player.

Soccer Game: The soccer game (FIG. 13, FIG. 14) may aim to encourage the player to reach out of their base of support in a static stance or more dynamic stepping stance. A game may have the player guarding a soccer goal, with soccer balls that may be aimed at nine points on the screen (top left, middle left, bottom left, top right, middle right, bottom right, top center, middle center and bottom center). The player and clinician may save a player profile to determine the position of the nine points (FIG. 13, e.g. to determine how ‘far’ to the left/right/top/bottom the ball will be directed, depending on the level of ability/reach of the player). The clinician may then control the rate and order of the soccer balls as they are presented to the player during the game-based task. The player may need to try to save as many balls as possible from entering the goal area.

### Example

### Card Match Game Design and Implementation

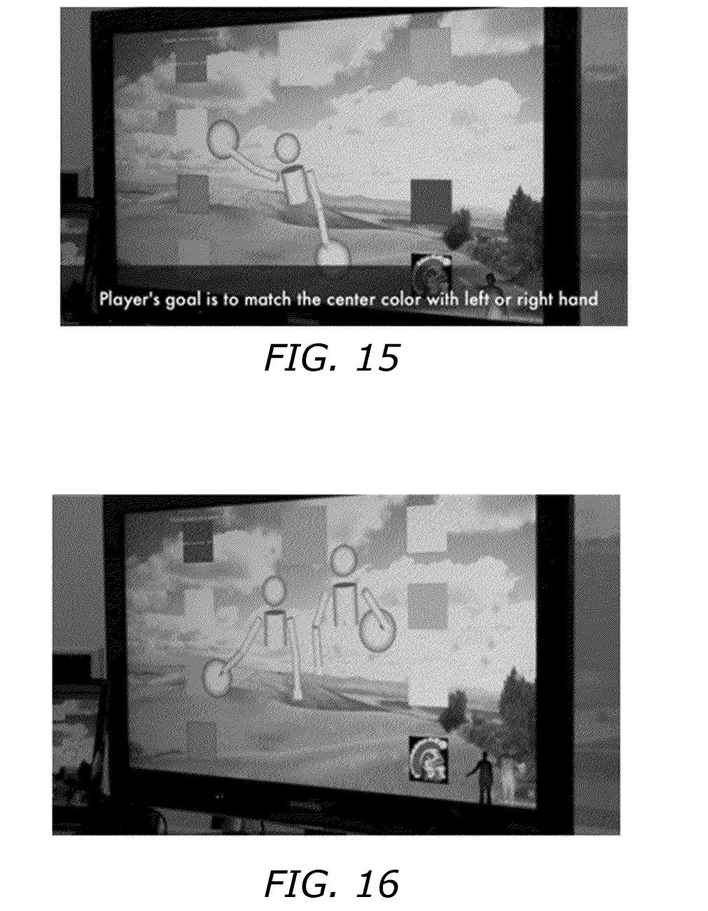

FIG. 15 illustrates an example of a one player version of a card-match game. FIG. 16 illustrates an example of a two player version of a card-match game.

Card-match Game: The card-match game may be a one or two player game (FIGS. 15 and 16 respectively) in which a ‘target’ card is presented on the center of the screen and the player(s) are encouraged to place their avatar's hand over the card that matches the ‘target’ card. A game may involve colored ‘target’ cards, however, the game may instead involve more cognitively challenging ‘targets,’ such as numbers and/or patterns.

### Example

### Shoulder Game Design and Implementation

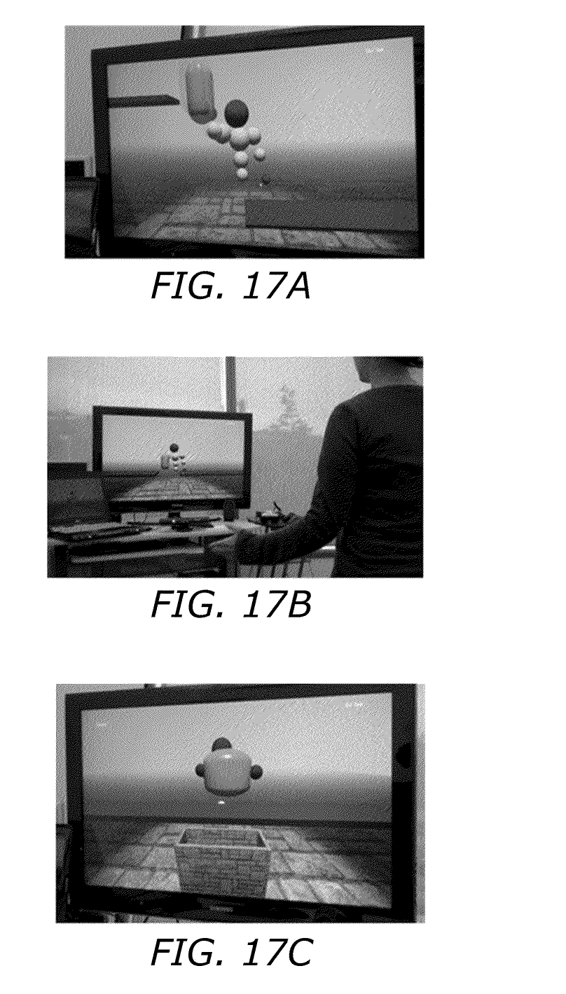

FIGS. 17A-17C illustrate examples of a shoulder strengthening exercise in a shoulder game.

Shoulder Game: The shoulder game is an example of a game that may incorporate strengthening exercises into a game-based tool (FIG. 17). Three exercises may be presented separately and may encourage the player to perform the shoulder strengthening exercises with appropriate body position and control.

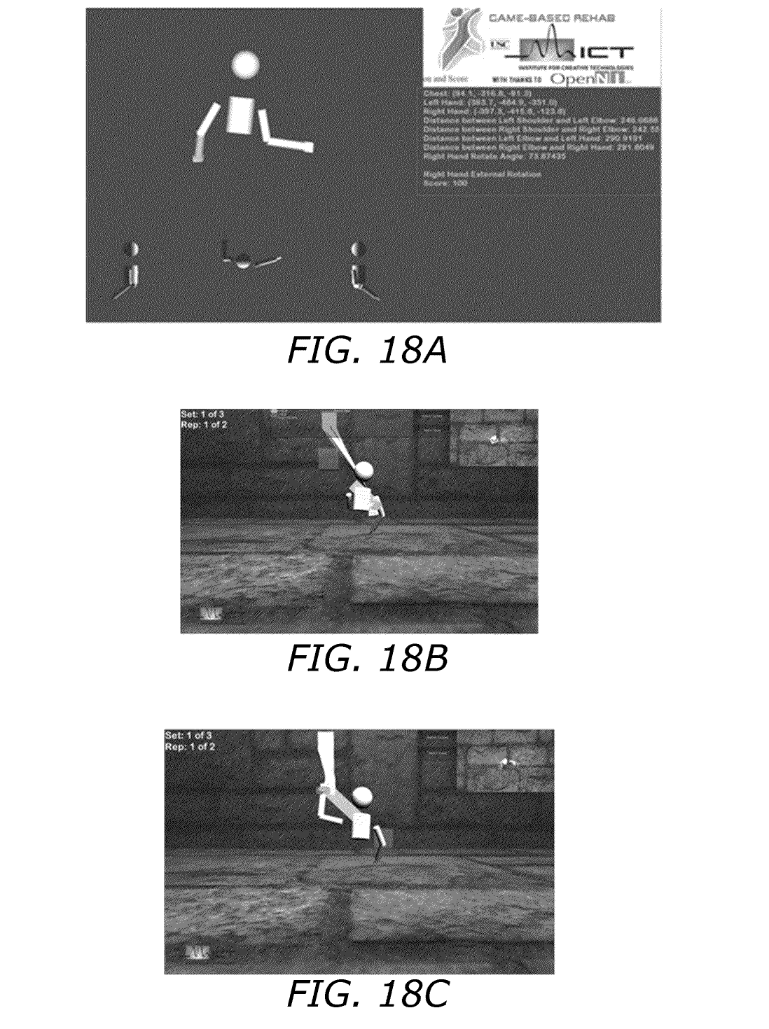

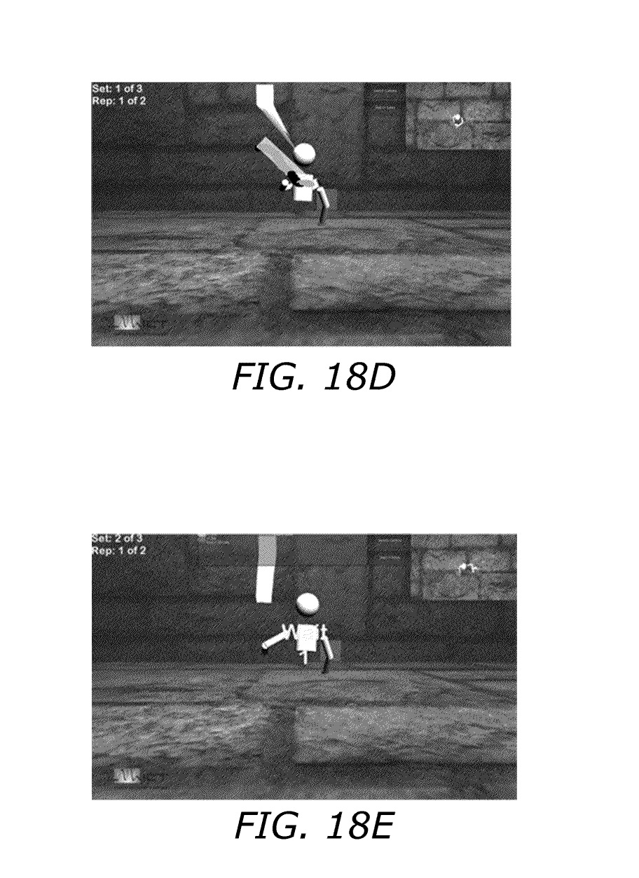

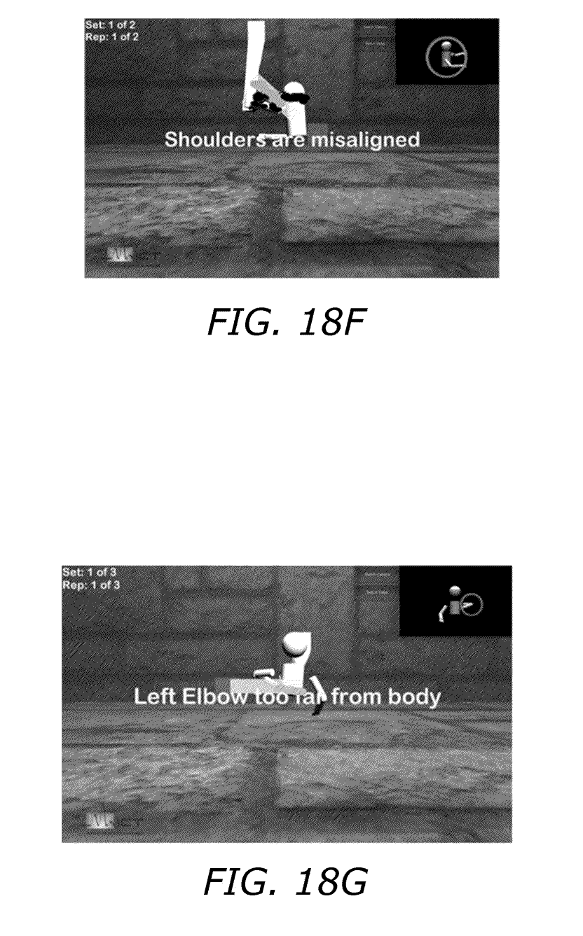

FIG. 18A-18G illustrate an example of a series of performance feedback. Light dotted areas may indicate correct movement location. The dark areas may indicate incorrect movement location. When the user has their body positioned incorrectly, the system may provide real time feedback, such as in the top right hand corner and using text in the center of the screen to instruct the user how to change their performance.

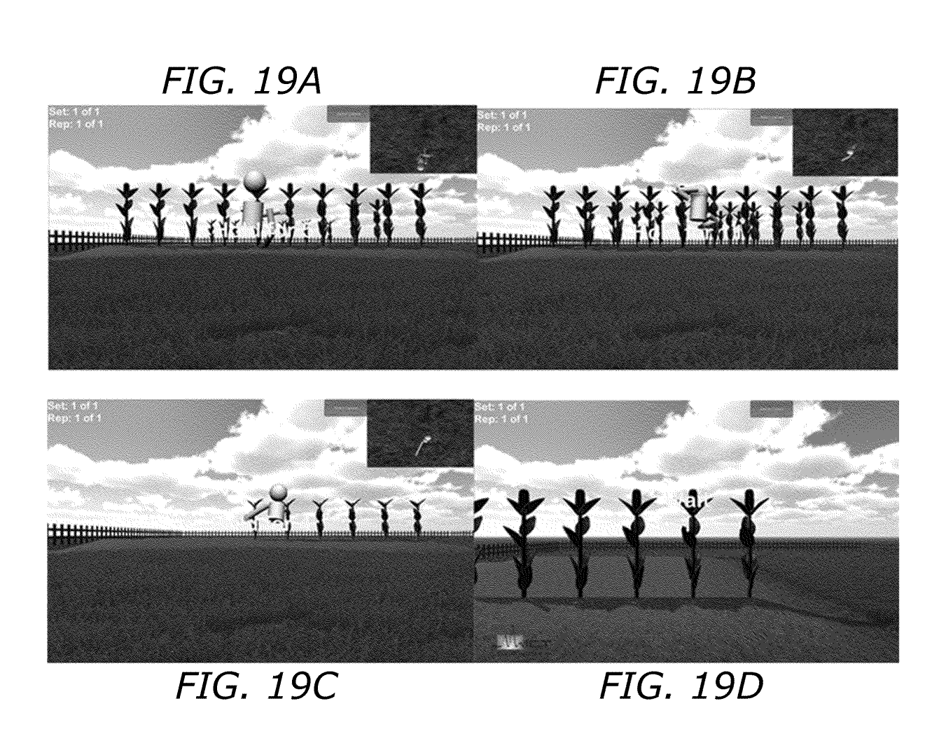

FIGS. 19A-19D illustrate an example of changes to the environment. For example, the graphics may be changed from those in FIG. 17 and FIG. 18 to the more game-like graphics of a farm setting in which the user's exercise performance may control actions in the game, such as planting, watering and cutting plants.

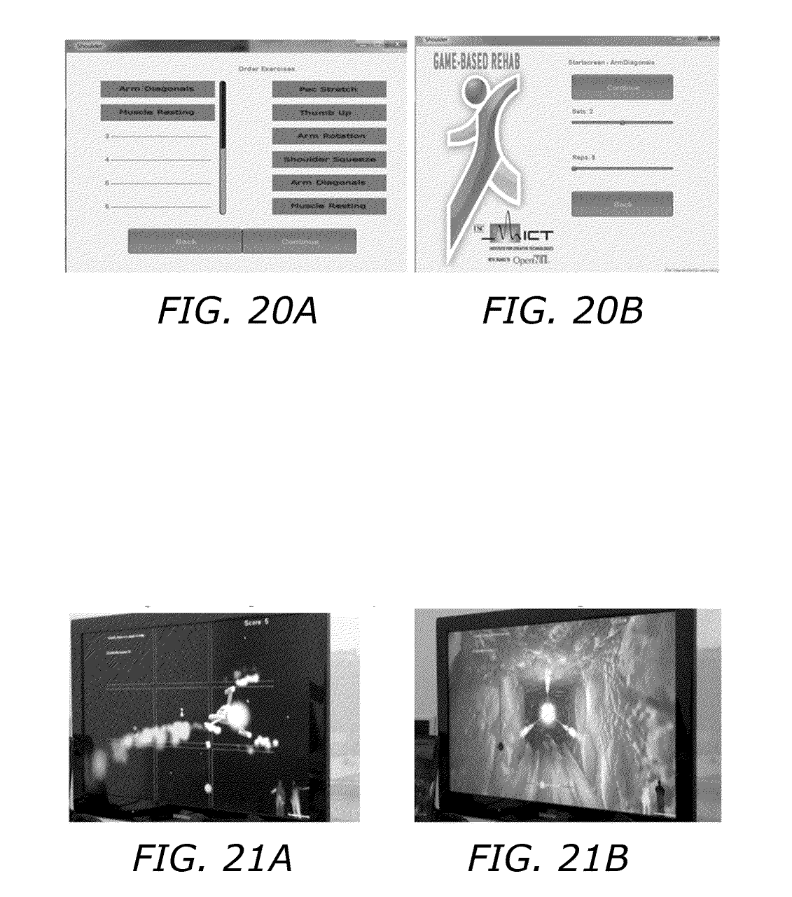

FIGS. 20A-B illustrate examples of how a game may be tailored at several points. The user may choose which exercises they want to perform and the order of the exercises. The user may also choose the number of sets and the number of repetitions of the exercises/tasks. These exercises and tasks may include stretches, strengthening exercises, functional tasks, endurance exercises, break down on movements to practice separately, and an option to put tasks together to complete more complex tasks.

### Example

### Star Tour Game

Star Tour Game: A star tour game may be a one or two player game in which the player(s) use their body movement (arms only or whole body-squat/stand/step left/step right) to control the flight path of a space shuttle in order to collect objects and successfully travel through a tunnel. In a two player version of the game, one player may control the vertical direction of the space shuttle and the second player may control the horizontal direction of the space shuttle. The location of the targets, the type of targets, and the speed of targets and the ship may be changed within the game. User input controls may also be changed.

FIGS. 21A-B illustrate two levels in an example Star tour game.

### Example

### Shopping Task

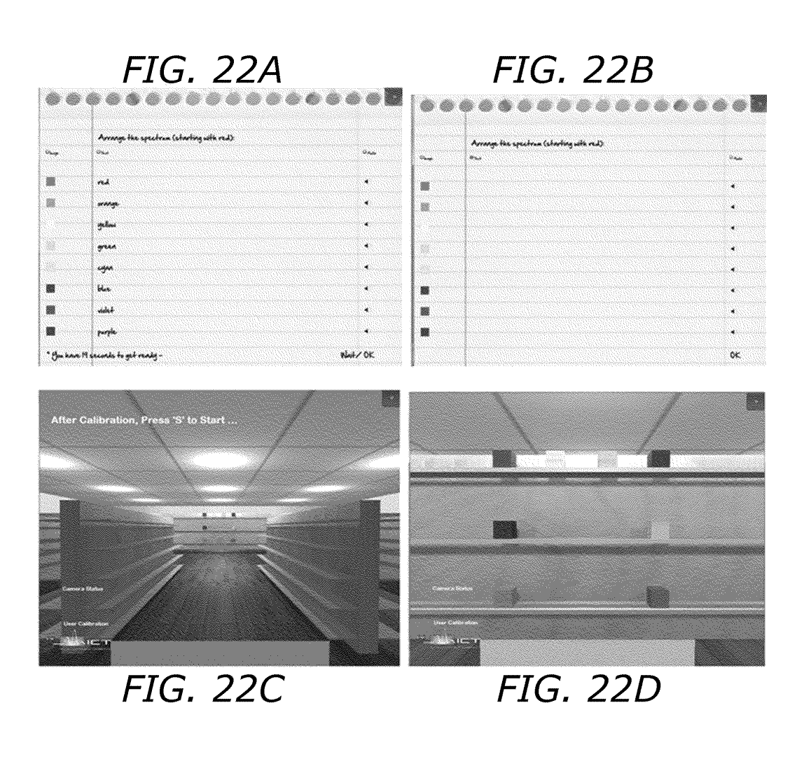

FIGS. 22A-22D illustrate examples of a shopping game. FIGS. 22A-22B illustrate examples of shopping tasks, and FIGS. 22C-22D illustrate examples of task scenes.

Shopping Task: The shopping task (See FIG. 22) may allow the player(s) to use their body movement (arms only or whole body-squat/stand/step left/step right) to control their path through a grocery store. The task may be set up in a number of different ways. The player may be asked to remember a list of items they must collect in the store. The player may also be given the option to purchase items—spending a certain amount of money they are given. The shopping list may be provided in images, text and audio, or a combination of those forms, depending on how the task is to be structured.

### Example

### Mystic Isle—Physical and Cognitive Challenge Task

Mystic Isle: A software application that may allow the clinician/trainer/third party and/or human subject to perform customized activities that challenge physical and cognitive constructs. The software may provide a flexible user interface that allows the human subject, trainer and/or clinician to modify tasks and/or modify calibration features to change the level of challenge of the task/activity/exercise. The software may allow the human subject and/or trainer and/or clinician and/or third party to create a customized set of activities to be completed by the human subject. This customized set of activities may contain a series of tasks to be performed in order and may be modified over time. The software application may provide options of tasks/activities that contain functional and/or abstract features that may be used as assessment and/or training tools for physical and cognitive domains.

### Example

### Wii Fit Games for Weight Shift Training

A game may be developed using the Panda 3D game engine and/or using the Unity Game Engine. The game may be played on a PC and may use the Nintendo® WiiFit™ balance board as the interaction device.

The balance board may contain multiple sensors located on its bottom corners to measure and calculate the center of pressure changes. When the player moves their body on the balance board, rather than being transmitted to the Nintendo® Wii™ console, the data from the sensors may be transmitted to a computer, such as by using Bluetooth. The data may include multiple kinds of information, such as the weight from each of the four corners of the board, the total weight, and the x and y coordinates of the center of pressure.

Within the game, the player may calibrate their range of motion or may calibrate a required task difficulty (in standing or sitting with their feet on the board, using their hands on the board, a range of other positions, such as by using foam mats and/or fit balls).

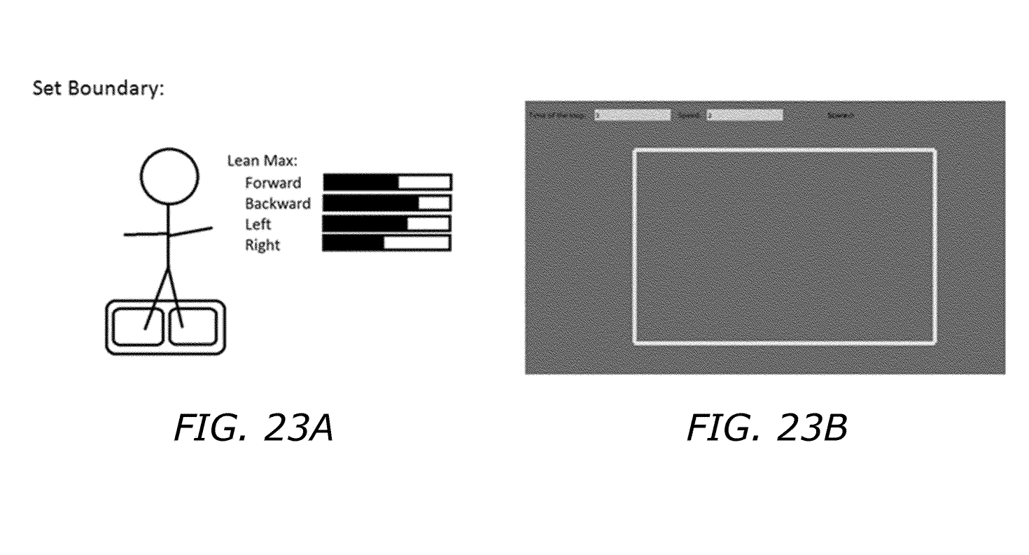

FIG. 23A illustrates an example of a boundary box formed by a user that serves as a calibrated area of movement. The user may shift their weight forward on the pressure board as far as they can or as required by their exercise goals. This movement may set the top boundary line. The user may shift their weight to the left on the pressure board as far as they can or as required by their exercise goals. This movement may set the left boundary line. The user may shift their weight right on the pressure board as far as they can or as required by their exercise goals. This movement may set the right boundary line. The user may shift their weight backward on the pressure board as far as they can or as required by their exercise goals. This movement may set the bottom boundary line.

FIGS. 23A-B illustrate an example of a calibration process. A goal of the tasks developed in these systems may involve the user controlling a control object and avoiding or collecting target objects or obstacles. These targets and obstacles may be placed in such a way that the user has to shift weight from one leg to another in a controlled pattern and maintain that weight shift for a period of time.

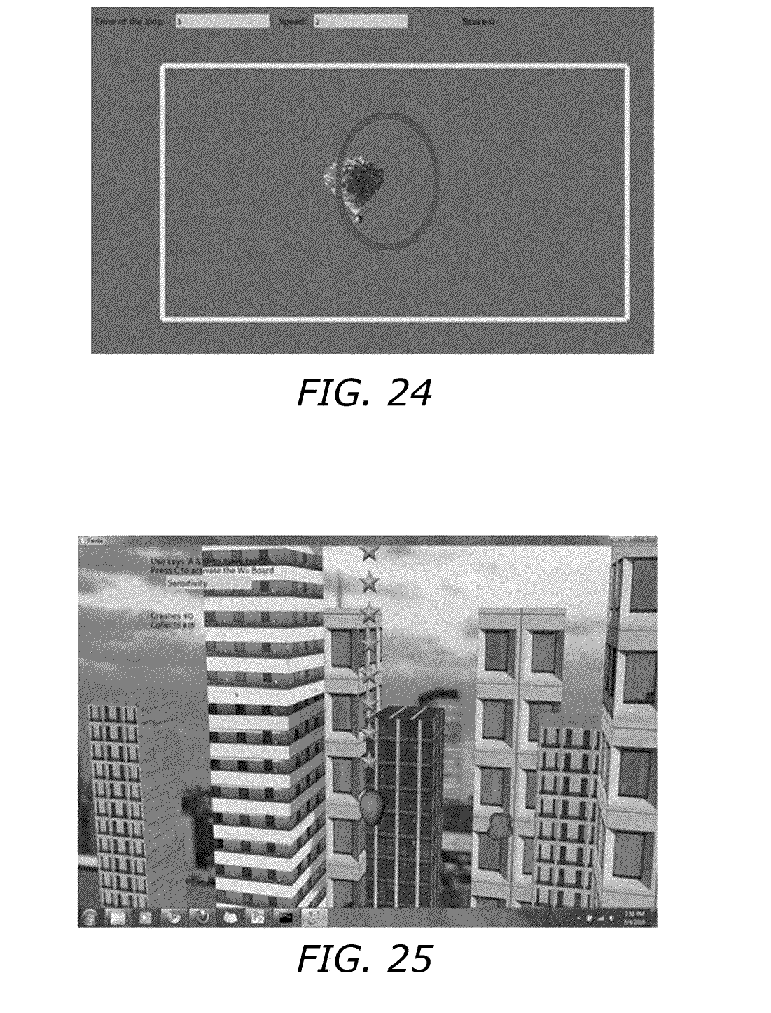

FIG. 24 illustrates an example of a game task in which a user is asked to control a circle or shaped object on a screen, using body motion. The user may be asked to track the target object on the screen (may be stationary or moving) using their body movements to control the circle-shaped object. The target object may be set to perform the required actions within the user's calibrated boundary box area.

FIG. 25 illustrates an example of a game task in which a user is asked to control a balloon on a screen. The user may be asked to collect the target objects on the screen (number, placement, location, and speed of target objects may be controlled by the user or clinician) and avoid other distraction objects on the screen (number, placement, location, and speed of target objects may be controlled by the user or clinician). The target objects may be set to perform the required actions within the user's calibrated boundary box area. The game may have scoring for the number of objects collected and the number of unwanted collisions with rocks. Different sounds may be provided when the balloon connects with the stars and collides with the rocks. The balloon may not be damaged if it collides with the rock, so as not to discourage patients whilst learning to play the game. This may reduce the number of stop/start delays that occur during current commercial Nintendo® WiiFit™ games when the task is not achieved.

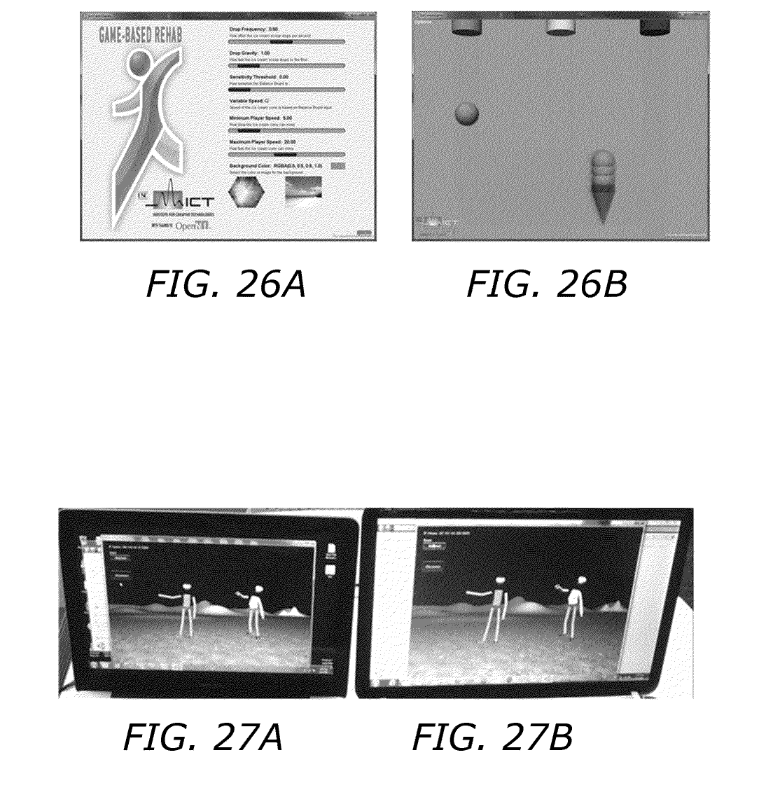

FIGS. 26A-B illustrate an example of a game task in which a user is asked to control an ice cream cone on a screen. The user may be asked to collect the target objects on the screen (number, placement, location, and speed of target objects may be controlled by the user or clinician) and avoid other distraction objects on the screen (number, placement, location, and speed of target objects may be controlled by the user or clinician). The target objects may be set to perform the required actions within the user's calibrated boundary box area. The background color may be altered using a color tool or a background picture may be uploaded. Graphics of control objects and target objects may be changed to alter the theme of the task.

### Rendering Multiple Body-Tracked Avatars Across a Network for Telemedicine Applications

Multiple full-body-tracked avatars may be rendered across a network utilizing the computer vision capabilities of the Microsoft Kinect. Multiple networked clients may be connected to a server machine. For example, one server and one client may represent the potential scenario of a therapist interacting with a patient. The Microsoft Kinect SDK and the Unity game engine may be utilized for this implementation. Multiple users may be able to interact with each other's avatars through body movement, in real time, across any network. The game may allow a therapist to demonstrate exercises to a patient and, additionally, to be provided with visual feedback as to how well the patient is doing the exercises.

FIGS. 27A-B illustrate an example of multiple body-tracked avatars across a network being rendered in a single display. This may be used for telemedicine. Here, a therapist (server) and a patient (client) may be interacting together while being in separate locations. The game may be calculating and providing visual feedback in the form of texture coloring on the arms, to show how well the patient is following the exercises provided by the therapist.

To setup this game, a Unity network class may be initialized, ports may be set, and game names may be given for the Master Server to use. NAT punch through may be enabled so that the networking may work on all networks, private and public.

When creating an object in Unity that is intended to be synchronized across a network, Network.Instantiate( ) may be called. Additionally, a NetworkViewlD may be associated with the object to be instantiated. Once this is done, calling Network.Instanitate on the game object may create it both on the local computer, as well as on all connected computers. All synchronization of this object may be handled automatically through Unity's networking implementation. If the avatar is instantiated on the machine that acts as the server, the associated data stream may be set for writing. All data may be sent across the network, to be synchronized with all the other clients. If the object was instantiated on a client computer, then the stream may be set for reading to receive updates from the server about the position of avatar joints. This data transfer may be done for all joints of the implemented avatar such that the Kinect's data about positions of limbs, head and trunk are transferred across the network and synchronized for all connected PCs.

The update of avatar limbs may be being handled by a set of custom dlls (UnityInterface.dll and UnityKinectPlugin.dll) that communicate with the Kinect for Windows SDK. This implementation may be identical to any of the other applications that have been described herein, such that the network implementation may be added to all applications (e.g. JewelMine/Mystic Isle) without much implementation effort.

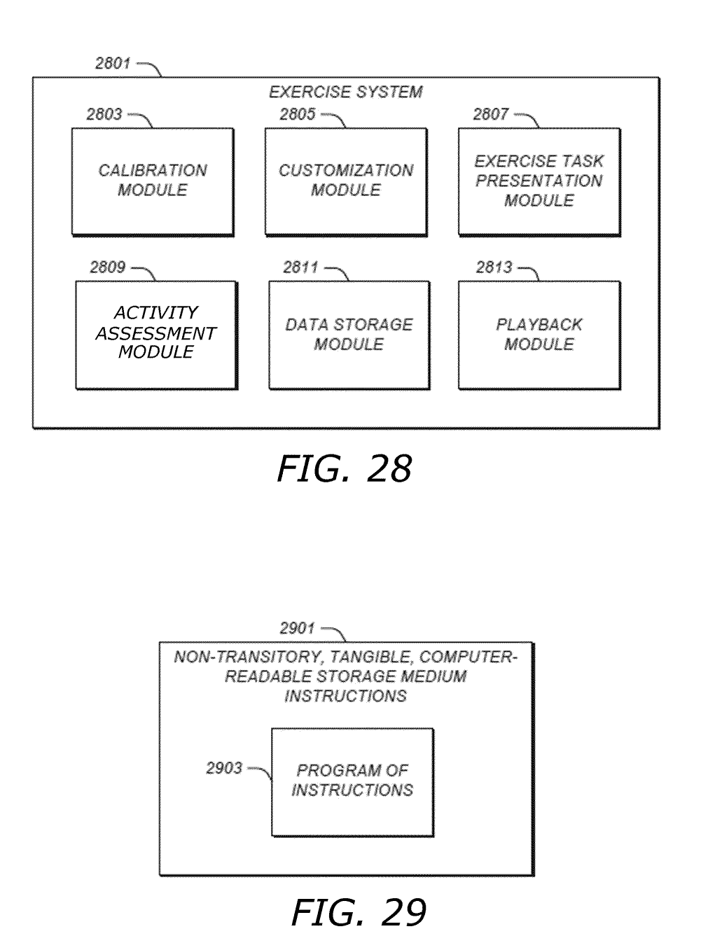

FIG. 28 illustrates an example of modules in an exercise system 2801. As illustrated in FIG. 28, the exercise system 2801 may include a calibration module 2803, a customization module 2805, an exercise task presentation module 2807, an exercise assessment module 2809, a data storage module 2811, and a playback module 2813.

The exercise system 2801 in conjunction with its various modules may be configured to implement one or more of the various calibration, customization, game presentation, and game assessment tasks that have been described above.

For example, the calibration module 2803 may measure a range of limb and trunk movements made by a human subject during a calibration phase of the exercise system 2801. The calibration module 2803 may determine a maximum range of movement made by the human subject during the calibration phase. To facilitate this, the calibration module 2803 may include one or more cameras configured to capture the image of the human subject moving and/or other movement capture devices, such as position sensors and/or accelerometers. The calibration module 2803 may include signal processing and image recognition technology configured to decipher between movement of the human subject and other stationary or moving objects in the scene.

The customization module 2805 may customize pre-determined performance criteria based on the measured range of limb and trunk movements. The customization module 2805 may adjust the pre-determined performance criteria during an exercise phase so as to encourage the human subject to continue the exercise task.

The movement task presentation module 2807 may present an exercise task to a human subject for performance during the exercise or activity phase. The exercise task presentation module 2807 may track the movement of the human subject during the exercise phase, display an avatar that moves in substantial synchronism with the human subject during the exercise phase, and smoothen the movements of the displayed avatar so as to substantially stop momentary lapses in the tracking of the movement of the human subject from causing sudden jumps in the movement of the display avatar.

The task presentation module 2807 may adjust the height and limb length of the avatar to reflect the height and limb length of the human subject.

The task presentation module 2807 may receive information concerning the movement of other human subjects that are not at the same location as the human subject and, for each of such other human subjects, displaying an additional avatar that moves in substantial synchronism with the other human subject.

The movement task activity assessment module 2809 may assess the human subject's performance of the exercise task during the exercise phase, based on the pre-determined performance criteria. The activity assessment module 2809 may have a viewing window within it which monitors movement of the human subject and that locks onto the human subject so that the activity assessment module 2809 does not erroneously confuse movement of another person or persons within the viewing window as movement of the human subject.

The data storage module 2811 may store information indicative of the human subject's movement during the calibration phase and/or performance of the movement task during the activity phase. The playback module 2813 may playback the stored information after the calibration phase and/or the activity phase in response to a playback request.

FIG. 29 illustrates an example of a non-transitory, tangible, computer-readable storage medium 2901 containing a program of instructions 2903. The program of instructions 2903 may cause a computer system running the program of instructions to implement a system of any of the types discussed above.

Unless otherwise indicated, the system that has been discussed herein, as well as each of its modules, are implemented with a computer system configured to perform the functions that have been described herein for the system and each module. A separate computer system may be used for each module. Conversely, one or more of the modules may be part of the same computer system. Each computer system includes one or more processors, tangible memories (e.g., random access memories (RAMs), read-only memories (ROMs), and/or programmable read only memories (PROMS)), tangible storage devices (e.g., hard disk drives, CD/DVD drives, and/or flash memories), system buses, video processing components, network communication components, input/output ports, and/or user interface devices (e.g., keyboards, pointing devices, displays, microphones, sound reproduction systems, and/or touch screens).

Each computer system may be a desktop computer or a portable computer, such as a laptop computer, a notebook computer, a tablet computer, a PDA, a smartphone, or part of a larger system, such a vehicle, appliance, and/or telephone system.

Each computer system may include one or more computers at the same or different locations. When at different locations, the computers may be configured to communicate with one another through a wired and/or wireless network communication system.

Each computer system may include software (e.g., one or more operating systems, device drivers, application programs, and/or communication programs). When software is included, the software includes programming instructions and may include associated data and libraries. When included, the programming instructions are configured to implement one or more algorithms that implement one or more of the functions of the computer system, as recited herein. The description of each function that is performed by each computer system also constitutes a description of the algorithm(s) that performs that function.

The software may be stored on or in one or more non-transitory, tangible storage devices, such as one or more hard disk drives, CDs, DVDs, and/or flash memories. The software may be in source code and/or object code format. Associated data may be stored in any type of volatile and/or non-volatile memory. The software may be loaded into a non-transitory memory and executed by one or more processors.

The components, steps, features, objects, benefits, and advantages that have been discussed are merely illustrative. None of them, nor the discussions relating to them, are intended to limit the scope of protection in any way. Numerous other embodiments are also contemplated. These include embodiments that have fewer, additional, and/or different components, steps, features, objects, benefits, and advantages. These also include embodiments in which the components and/or steps are arranged and/or ordered differently.

For example, although having been primarily discussed for encouraging and managing exercise, the systems and methods that have been described may also be used to train, test, rehabilitate, and/or guide movements for other purposes, such as for musical instrument training, sports training, and/or surgery training or rehearsal.

Although having been primarily discussed as having a clinician, trainer or human subject control the customization of the system, the customization may be performed by the human subject or a third party.

The software may contain a user interface that allows the human subject and/or a trainer and/or a clinician and/or a third party to modify the tasks/activities/exercises on a number of levels including but not limited to length of task, order of task, number and/or form of target objects, location of target objects, behavior of target objects. Location of target objects may be customized through the use of calibration data and/or task settings. A series of tasks may be combined to be performed in a customized order within a session.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

All articles, patents, patent applications, and other publications that have been cited in this disclosure are incorporated herein by reference.

The phrase “means for” when used in a claim is intended to and should be interpreted to embrace the corresponding structures and materials that have been described and their equivalents. Similarly, the phrase “step for” when used in a claim is intended to and should be interpreted to embrace the corresponding acts that have been described and their equivalents. The absence of these phrases from a claim means that the claim is not intended to and should not be interpreted to be limited to these corresponding structures, materials, or acts, or to their equivalents.

The scope of protection is limited solely by the claims that now follow. That scope is intended and should be interpreted to be as broad as is consistent with the ordinary meaning of the language that is used in the claims when interpreted in light of this specification and the prosecution history that follows, except where specific meanings have been set forth, and to encompass all structural and functional equivalents.

Relational terms such as “first” and “second” and the like may be used solely to distinguish one entity or action from another, without necessarily requiring or implying any actual relationship or order between them. The terms “comprises,” “comprising,” and any other variation thereof when used in connection with a list of elements in the specification or claims are intended to indicate that the list is not exclusive and that other elements may be included. Similarly, an element preceded by an “a” or an “an” does not, without further constraints, preclude the existence of additional elements of the identical type.

None of the claims are intended to embrace subject matter that fails to satisfy the requirement of Sections 101, 102, or 103 of the Patent Act, nor should they be interpreted in such a way. Any unintended coverage of such subject matter is hereby disclaimed. Except as just stated in this paragraph, nothing that has been stated or illustrated is intended or should be interpreted to cause a dedication of any component, step, feature, object, benefit, advantage, or equivalent to the public, regardless of whether it is or is not recited in the claims.

The abstract is provided to help the reader quickly ascertain the nature of the technical disclosure. It is submitted with the understanding that it will not be used to interpret or limit the scope or meaning of the claims. In addition, various features in the foregoing detailed description are grouped together in various embodiments to streamline the disclosure. This method of disclosure should not be interpreted as requiring claimed embodiments to require more features than are expressly recited in each claim. Rather, as the following claims reflect, inventive subject matter lies in less than all features of a single disclosed embodiment. Thus, the following claims are hereby incorporated into the detailed description, with each claim standing on its own as separately claimed subject matter.

The invention claimed is:

1. A system comprising:
a calibration module that has a configuration that measures a range of limb and trunk movements made by a human subject during a calibration phase of the system;
a customization module that has a configuration that customizes pre-determined performance criteria based on the measured range of limb and trunk movements;
a task presentation module that has a configuration that presents a movement task to the human subject for performance during an activity phase; and
an activity assessment module that has a configuration that assesses the human subject's performance of that movement task during the activity phase based on the pre-determined performance criteria.

2. The system of claim 1 wherein the activity assessment module has a configuration that has a viewing window within which it monitors movement of the human subject and that locks onto the human subject so that the activity assessment module does not erroneously confuse movement of another person or persons within the viewing window as movement of the human subject.

3. The system of claim 1 wherein the customization module adjusts the pre-determined performance criteria during the activity phase so as to encourage the human subject to continue the movement task.

4. The system of claim 1 further comprising:
a data storage module that has a configuration that stores information indicative of the human subject's performance of the movement task during the activity phase; and
a playback module that has a configuration that plays back the stored information after the activity phase.

5. The system of claim 1 further comprising:
a data storage module that has a configuration that stores information indicative of the human subject's movement during the calibration phase; and
a playback module that has a configuration that plays back the stored information after the calibration phase.

6. The system of claim 1 wherein the activity assessment module determines the velocity of limb and trunk movement made by the human subject during the activity phase and utilizes this determined velocity in the assessment.

7. The system of claim 1 wherein the activity assessment module determines a range of movement made by the human subject during the activity phase and utilizes this determined range of movement in the assessment.

8. The system of claim 7 wherein:
the calibration module has a configuration that determines a maximum range of movement made by the human subject during the calibration phrase; and
the activity assessment module has a configuration that assesses the human subject's performance of the movement task during the activity phase to have been satisfactory when the determined range of movement made by the human subject during the activity phase meets or exceeds a pre-determined portion that is less than 100% of the maximum determined range of movement.

9. The system of claim 1 wherein the movement task presentation module has a configuration that:
tracks the movement of the human subject during the activity phase;
displays an avatar that moves in substantial synchronism with the human subject during the activity phase; and
smoothens the movements of the displayed avatar so as to substantially stop momentary lapses in the tracking of the movement of the human subject from causing sudden jumps in the movement of the displayed avatar.

10. The system of claim 1 wherein the movement task presentation module has a configuration that:
tracks the movement of the human subject during the activity phase;
displays an avatar that moves in substantial synchronism with the human subject during the activity phase; and
adjusts the height and limb length of the avatar to reflect the height and limb length of the human subject.

11. The system of claim 1 wherein the movement task presentation module has a configuration that:
tracks the movement of the human subject during the activity phase;
displays an avatar that moves in substantial synchronism with the human subject during the activity phase;
receives information concerning the movement of other human subjects that are not at the same location as the human subject; and
for each of such other human subjects, displays an additional avatar that moves in substantial synchronism with the other human subject.

12. The system of claim 1 wherein the movement task is an exercise task.

13. A non-transitory, tangible, computer-readable storage media containing a program of instructions that cause a computer system running the program of instructions to:
measure a range of limb and trunk movements made by a human subject during a calibration phase of the computer system;
customize pre-determined performance criteria based on the measured range of limb and trunk movements.
present an movement task to the human subject for performance during an activity phase; and
assess the human subject's performance of that movement task during the activity phase based on the pre-determined performance criteria.

14. The storage media of claim 13 wherein the program of instructions causes the computer system to lock onto the human subject within a viewing window so that the computer system does not erroneously confuse movement of another person or persons within the viewing window as movement of the human subject.

15. The storage media of claim 13 wherein the program of instructions causes the computer system to adjust the pre-determined performance criteria during the activity phase so as to encourage the human subject to continue the movement task.

16. The storage media of claim 13 wherein the program of instructions causes the computer system to:
store information indicative of the human subject's performance of the movement task during the activity phase; and
play back the stored information after the activity phase.

17. The storage media of claim 13 wherein the program of instructions causes the computer system to:
store information indicative of the human subject's movement during the calibration phase; and
plays back the stored information after the calibration phase.

18. The storage media of claim 13 wherein the program of instructions causes the computer system to determine the velocity of limb and trunk movement made by the human subject during the activity phase and utilize this determined velocity in the assessment.

19. The storage media of claim 13 wherein the program of instructions causes the computer system to determine a range of movement made by the human subject during the activity phase and utilize this determined range of movement in the assessment.

20. The storage media of claim 19 wherein the program of instructions causes the computer system to:
determine a maximum range of movement made by the human subject during the calibration phrase; and
assess the human subject's performance of the movement task during the activity phase to have been satisfactory when the determined range of movement made by the human subject during the activity phase meets or exceeds a pre-determined portion that is less than 100% of the maximum determined range of movement.

21. The storage media of claim 13 wherein the program of instructions causes the computer system to:
track the movement of the human subject during the activity phase;
display an avatar that moves in substantial synchronism with the human subject during the activity phase; and
smoothen the movements of the displayed avatar so as to substantially stop momentary lapses in the tracking of the movement of the human subject from causing sudden jumps in the movement of the displayed avatar.

22. The storage media of claim 13 wherein the program of instructions causes the computer system to:
track the movement of the human subject during the activity phase;
display an avatar that moves in substantial synchronism with the human subject during the activity phase; and
adjust the height and limb length of the avatar to reflect the height and limb length of the human subject.

23. The storage media of claim 13 wherein the program of instructions causes the computer system to:
track the movement of the human subject during the activity phase;
display an avatar that moves in substantial synchronism with the human subject during the activity phase;
receive information concerning the movement of other human subjects that are not at the same location as the human subject; and
for each of such other human subjects, display an additional avatar that moves in substantial synchronism with the other human subject.

24. The storage media of claim 13 wherein the movement task is an exercise task.
